(19) United States
(12) Patent Application Publication
(10) Pub. No.: US 2010/0028991 A1
(43) Pub. Date: Feb. 4, 2010

(54) Asymmetric compound parabolic concentrator and related systems

(76) Inventor: Joe McCall, Sandy Springs, GA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 12/321,062

(22) Filed: Jan. 14, 2009

Related U.S. Application Data
(60) Provisional application No. 61/020,927, filed on Jan. 14, 2008.

(51) Int. Cl. C12M 3/00; F21V 11/00; F21V 9/12; G02B 5/10

(52) U.S. Cl. 435/292.1; 435/289.1; 362/351; 362/318; 359/853

(57) ABSTRACT

Various embodiments disclosed herein provide for an asymmetric compound parabolic concentrator (ACPC), a liquid light guide, and an algae mixer. These embodiments may be used singularly or in combination, for example, as part of a bioreactor. In various embodiments an ACPC may be used to capture light without the aid of motors or positioning. As such, the ACPC may be used despite seasonal solar variations. The illuminating light guide may be used to guide light, for example, from an exit aperture of an ACPC, through walls substantially perpendicular to the entrance window of the illuminating light guide. In some embodiments, the illuminating light guide may provide substantially uniform light distribution through a vertical profile. Other embodiments include, for example, an algae mixer and bioreactors.

### CROSS-REFERENCES TO RELATED APPLICATIONS

This application claims benefit to U.S. Provisional Application No. 61/020,927, filed on Jan. 14, 2008.

### BACKGROUND

There is considerable interest in the development of renewable energy sources to replace petroleum-based fuels. It has been discovered that certain algae have a large oil or lipid content, and thus provide a source for the production of biodiesel. In some cases, algae may contain upwards of 40%, 50%, or even 70% oil by weight. However, there is a lack of efficient and cost-effective algal biomass production systems. Open pond technology is often expensive and susceptible to contamination. Current closed photobioreactors using fiber optic light transmission can be prohibitively expensive. Moreover, current solar light capturing devices require the use of motors in order to track the sun and provide solar energy to such things as algae production.

A need exists for improved devices and methods for generating biodiesel from algae and a need to provide motor-less light capturing devices. Preferably, such techniques would provide sufficient illumination to algae cultures to support growth. Further, these approaches should provide the required nutrients and gases to support algal growth. At least some of these objectives will be met by embodiments of the present invention.

### BRIEF SUMMARY

An asymmetric compound parabolic concentrator (ACPC) is provided according to some embodiments. In some embodiments, an ACPC includes first and second reflective surfaces, the first and second reflective surfaces each having a shape including both a linear portion and a parabolic portion. In some embodiments, the first reflective surface has a substantially straight top edge bounding a top portion of the first surface. The first reflective surface may also have a substantially straight bottom edge bounding a bottom portion of the first surface. The parabolic portion of the second reflective portion may be different from the parabolic portion of the first reflective surface. The second reflective surface may have a substantially straight bottom edge bounding a bottom portion of the second surface, and a substantially straight bottom edge bounding a bottom portion of the second surface. In some embodiments, the linear portion of the of the first reflective surface is contiguous with the bottom edge of the first linear surface. In some embodiments, the linear portion of the of the second reflective surface is contiguous with the bottom edge of the second reflective surface. In some embodiments, the top edge of the first surface and the top edge of the second surface are separated and define an entrance aperture. In some embodiments, the bottom edge of the first surface and the bottom edge of the second surface are separated and define an exit aperture. In some embodiments, the top edge of the first surface is substantially parallel with the top edge of the second surface, and the bottom edge of the first surface is substantially parallel with the bottom edge of the second surface.

In some embodiments, the concave side of the parabolic portion of the first surface faces the concave side of the parabolic portion of the second surface. In some embodiments, the asymmetric compound parabolic concentrator is tilted about 40° from vertical. In some embodiments, the first surface and/or the second surface and the area bounded by the exit aperture are substantially perpendicular. In some embodiments, the linear portion of the first surface is contiguous with the bottom edge of the first surface. In some embodiments, the linear portion of the second surface is contiguous with the top edge of the second surface. In some embodiments, the first reflective surface and the second reflective surface are immovably coupled with a light receiver.

A static ACPC is also provided that includes a first reflective surface having a shape including both a linear portion and a parabolic portion. The ACPC may also include a second reflective surface having a shape including both a linear portion and a parabolic portion. The parabolic portion of the second reflective portion may be different from the parabolic portion of the first reflective surface. The parabolic portion of the second reflective portion may be concave in the opposite direction as the parabolic portion of the first reflective portion. The first reflective surface and the second reflective surface may be immovably coupled with a light receiver. The static asymmetric compound parabolic concentrator concentrates solar radiation regardless of solar variations.

An illuminating light guide is also disclosed according to some embodiments. The illuminating light guide may include an entrance window, one or more layered transmissive sheets, and/or a light conductive material. The one or more layered transmissive sheets may include a portion contiguous with the entrance window. Each layered transmissive sheet may include a first substantially transparent layer and a second substantially transparent layer sandwiching a patterned air-gap layer. The ratio of the area covered by air-gaps to the area covered by non-air-gaps in the patterned air-gap layer may be greater near the entrance window. The light conductive material may be disposed within the illuminating light guide contiguous with a substantial portion of at least one of the one or more layered transmissive sheets.

Another illuminating light guide is disclosed according to another embodiment. In this embodiment, the illuminating light guide includes an entrance window configured to allow light to enter the light guide. The illuminating light guide may also include a first transmissive sheet contiguous and substantially perpendicular with the entrance window. The first transmissive sheet may include means for transmitting light from the entrance window through the first transmissive sheet with a substantially uniform vertical intensity gradient. A light conductive material may be contained within the illuminating light guide contiguous with a substantial portion of the first transmissive sheet.

Yet another illuminating light guide is provided according to another embodiment. In this embodiment, the illuminating light guide includes an entrance window configured to allow light to enter the light guide. The illuminating light guide may also include one or more layered transmissive sheets with a portion contiguous with the entrance window. Each of the one or more layered transmissive sheets includes a first substantially transparent layer and a second substantially transparent layer sandwiching a patterned contact layer. The patterned contact layer may be in contact with the first substantially transparent layer and the second substantially transparent layer according to a contact pattern that provides a greater density of contact further from the entrance window and a lesser density of contact near the entrance window. In this embodiment, the illuminating light guide also includes a light conductive material within the illuminating light guide contiguous with a substantial portion of at least one layered transmissive sheet.

An illuminating light guide is provided, according to another embodiment, including an entrance window, a first and a second transmissive sheet, and a light conductive material. The first transmissive sheet may be contiguous and substantially perpendicular with the entrance window. The second transmissive sheet may also be contiguous and substantially perpendicular with the entrance window, and substantially parallel with the first transmissive sheet. The light conductive liquid may be secured between the first transmissive sheet and the second transmissive sheet. The illuminating light guide may receive light through the entrance window and transmit the light through the first transmissive sheet and the second transmissive sheet with a substantially uniform vertical intensity gradient through both sheets.

An algae mixer is provided according to another embodiment. The algae mixer includes an actuator, and a plurality of scoops coupled with the actuator. Each scoop includes an opening. When a scoop is moved through a fluid by the actuator, a portion of the fluid is captured by the scoop and pressed through the opening at a speed greater than the speed of the scoop moving through the fluid. In some embodiments, each scoop includes a concave bottom portion and a convex side portion, and the opening is positioned contiguous with the junction of the bottom concave bottom portion and the convex side portion. In some embodiments, the actuator includes a belt and a motor, and the scoops are coupled with the belt. In some embodiments, the algae mixer is positioned near a light source. In some embodiments, at least one scoop includes a squeegee configured to clean a surface of the light source.

In one embodiment, the present disclosure provides for another ACPC. An ACPC may provide efficient trackless performance; that is, solar radiation may be concentrated at the ACPC exit aperture despite daily and seasonal variations in the sun's celestial position without requiring motorized tracking devices. In one embodiment, the ACPC may be tilted 40° from the vertical. Light may be collected between 10° and 70° from the vertical. In some embodiments, the parabolic surfaces may include linear portions. These linear portions may be found, for example, near the exit aperture of the ACPC.

In another embodiment, an illuminating light guide is provided. The illuminating light guide may include a trough filled with a clear fluid. The trough may be comprised of two parallel or semi-parallel layered sheets 115 that make up two sides of the illuminating light guide. The layered sheets 115 may comprise two sheets of transmissive material sandwiching a pattern of air-gaps. The air-gap pattern may provide a gradient of air-gaps from a high density of air-gaps near the top of the light guide to a low density of air-gaps near the bottom of the light guide. These layered sheets 115 may thus provide light transmission from the trough of water through the layered sheets 115 when light is incident on portions of the layered sheets 115 that do not include an air-gap. Because the proportion of light transmitting through the top of the light guide is greater than the portion near the bottom, the layered sheets 115 with an air-gap gradient provide nearly uniform light transmission over the surface of the sheets.

In yet another embodiment, the present disclosure provides for a mixer. In some embodiments the mixer may be an algae mixer. Algae may be located within an algae soup that includes water. The algae mixer, according to embodiments, may include a series of scoops coupled with an actuating device such as a belt that is configured to move the scoops through the algae. Each scoop may include a concave bottom surface, an open top, and/or a convex side surface. A small opening may be located near the juncture of the convex side surface and the concave bottom surface. As a scoop is moved through the algae soup, the algae soup is compressed as the shaped side and bottom portions force the algae soup through the small opening. The fluid exiting from the scoop through the small opening may exit at speeds greater than the speed of the scoop moving through the fluid. The scoops may be positioned such that they move algae that is originally far from an illuminating surface closer to an illuminating surface.

In yet another embodiment, the present disclosure also provides for an algae bioreactor that includes an ACPC, an illuminating light guide, an algae tank and an algae mixer. An ACPC may be coupled with the illuminating light guide. The ACPC receives light from the sun and directs the light into the illuminating light guide. The light enters the illuminating light guide at such an angle that all light rays are greater than the critical angle and thus undergo TIR at the surface of the light guide near an air-gap. Light exits the light guide through contacts through the layered sheets 115 and enters an algae tank. The algae tank may be mixed using the algae mixer described above.

Further areas of applicability of the present disclosure will become apparent from the detailed description provided hereinafter. It should be understood that the detailed description and specific examples, while indicating various embodiments, are intended for purposes of illustration only and are not intended to necessarily limit the scope of the disclosure.

### BRIEF DESCRIPTION OF THE DRAWINGS

The patent or application file contains at least one drawing executed in color. Copies of this patent or patent application publication with color drawings will be provided by the Office upon request and payment of the neccessary fee.

FIG. 1 shows an isometric view of an asymmetric compound parabolic concentrator coupled with an illuminating light guide according to one embodiment.

FIG. 2 shows a side view of an asymmetric compound parabolic concentrator coupled with an illuminating light guide according to one embodiment.

FIG. 3 shows a front view of an asymmetric compound parabolic concentrator coupled with an illuminating light guide according to one embodiment.

FIG. 4 shows a side view of an asymmetric compound parabolic concentrator showing exemplary angles and dimensions according to one embodiment.

FIGS. 5A and 5B show front views of illuminating light guides according to some embodiments.

FIG. 6 shows illumination results of an illumination light guide according to one embodiment.

FIG. 7 shows graphical illumination results of an illumination light guide according to one embodiment.

FIGS. 8A and 8B show illumination results of an illumination light guide with seasonally extreme incident light according to one embodiment.

FIG. 9 shows a side view of an algae trough situated between two illuminating light guides according to one embodiment.

FIG. 10 shows six illuminating light guides immersed in an algae trough according to one embodiment.

FIG. 11 shows one portion of an algae mixer according to one embodiment.

FIG. 12 shows an individual scoop of an algae mixer according to one embodiment.

FIG. 13 shows six illuminating light guides immersed in an algae trough with five recirculating algae mixers positioned between the illuminating light guides according to one embodiment.

FIG. 14 shows a side view of a non-horizontal asymmetric compound parabolic concentrator showing exemplary angles and dimensions according to one embodiment.

### DETAILED DESCRIPTION OF THE INVENTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It being understood that various changes may be made in the function and arrangement of elements without departing from the spirit and scope as set forth in the appended claims.

Various embodiments described herein are disclosed herein for renewable energy production. For instance, embodiments described herein may be used singularly or in combination with a bioreactor, and/or solar energy generation, etc.

For example, an asymmetric compound parabolic concentrator is disclosed in some embodiments that may collect light from the sun without the use of tracks despite seasonal variations in the sun's elevation. As another example, an illuminating light guide is provided that transmits light from an entrance aperture through at least one transmissive exit aperture that may be perpendicular with the entrance aperture. In some embodiments, the light exiting through the exit aperture does so relatively uniformly across a vertical gradient of the exit aperture. That is, for example, the amount of light exiting one portion of the exit aperture is relatively similar to the amount of light exiting through a second portion of the exit aperture despite being further removed from the entrance aperture. Yet another embodiment disclosed herein includes an algae mixer with a plurality of scoops (or cups) coupled with an actuated belt. These scoops, for example, may have a large opening and a smaller opening When a scoop is moved through a fluid by the actuator, a portion of the fluid is captured by the large opening of the scoop and pressed through the smaller opening at a speed greater than the speed of the scoop moving through the fluid. The embodiments described herein may be used singularly or in combination in any application.

### Asymmetric Compound Parabolic Concentrator

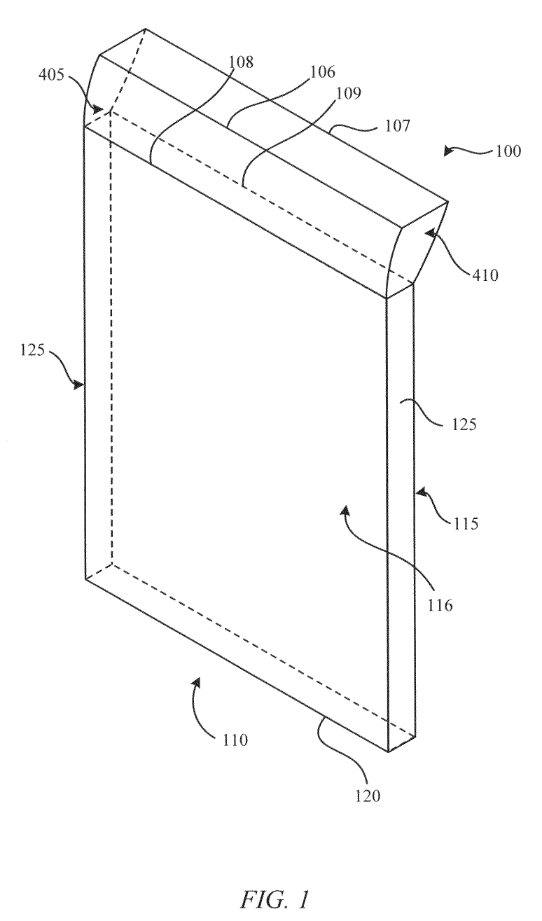

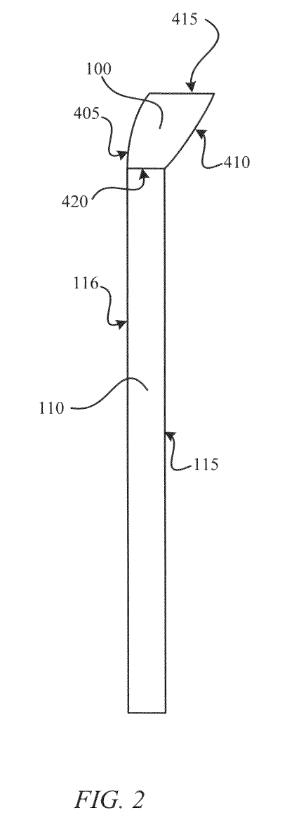

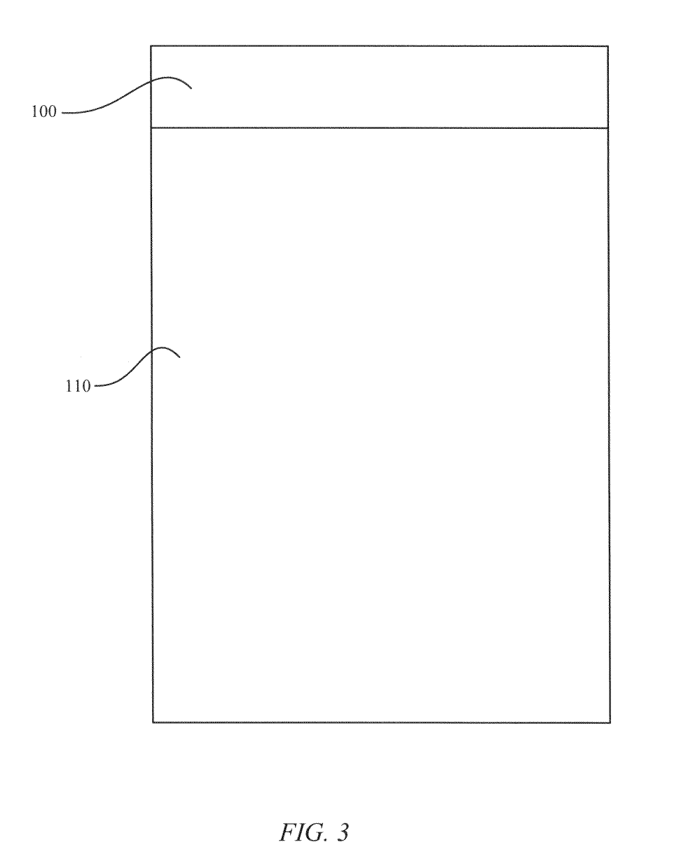

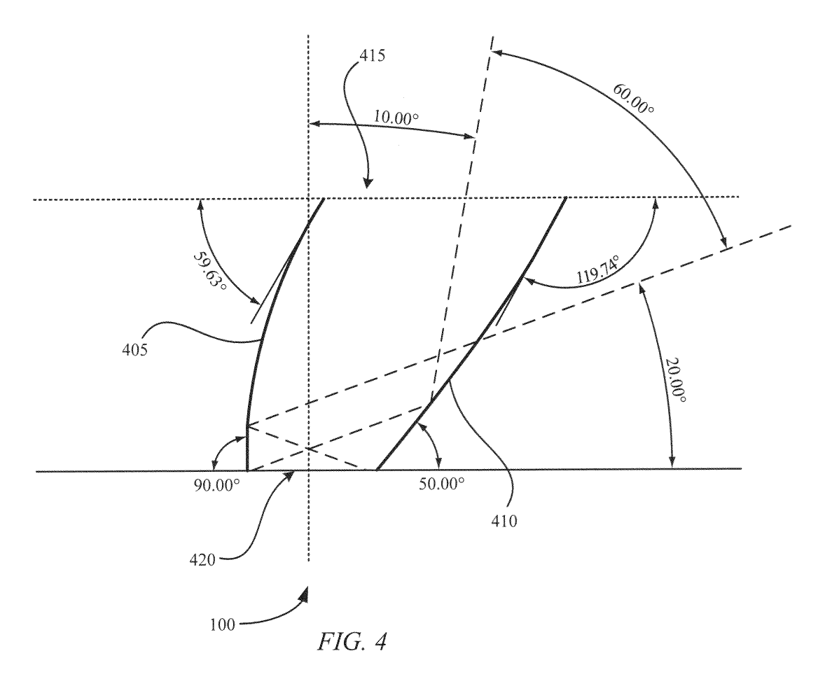

An asymmetric compound parabolic concentrator (ACPC) 100 as shown in FIGS. 1, 2, 3, and 4 is disclosed according to one embodiment. FIGS. 1, 2, and 3 show an ACPC 100 coupled with an illuminating light guide 110. ACPC 100 may include light reflective surfaces 405, 410 with shapes that include parabolic portions. The parabolic portions of the two surfaces have different shapes—hence the asymmetry. ACPC 100 may also include an entrance aperture (or entrance window) 415 and an exit aperture (or exit window) 420. ACPC 100 may collect light through entrance window 415 and reflect the light through to the exit aperture 420. The parabolic curves shaping the light reflective surfaces 405, 410 may be mathematically, geometrically, or visually different. That is the first reflective surface 405 may have a shape defined by one parabolic curve while the second reflective surface 410 may have a shape defined by a different parabolic curve or a different portion of the same parabolic curve. The shape of the two light reflective surfaces 405, 410 may also include substantially linear portions. As shown in FIG. 4, first reflective surface 405 includes a linear portion near exit aperture 420 and a parabolic portion near entrance aperture 415. Second reflective surface 410 includes a linear portion near exit aperture 420 and a parabolic portion near entrance aperture 415. Moreover, reflective surface 405 may also include a top straight edge 106 and a bottom straight edge 108, and reflective surface 410 may include a top straight edge 107 and bottom straight edge 109.

The dimensions and/or angles shown in FIG. 4 are examples only of one embodiment. In the embodiment shown in FIG. 4 solar light collects light at the entrance aperture over a ±30° range. In other embodiments light may be collected over other ranges, for example, ±20°, ±25°, ±35°, ±40°, or ±45°. ACPC 100 may also be configured with a 40° tilt from the vertical. Various other tilts may be used, for example, 5°, 10°, 15°, 20°, 25°, 30°, 35°, 45°, 50°, 55°, 60°, 65°, 70°, 80°, 85°, or 90°. Thus, with a 40° tilt and a ±30° light collection range, the output angular range may be ±70°. In some embodiments, first reflective surface 405 abuts with exit aperture 420 at 90° or other angles may be used.

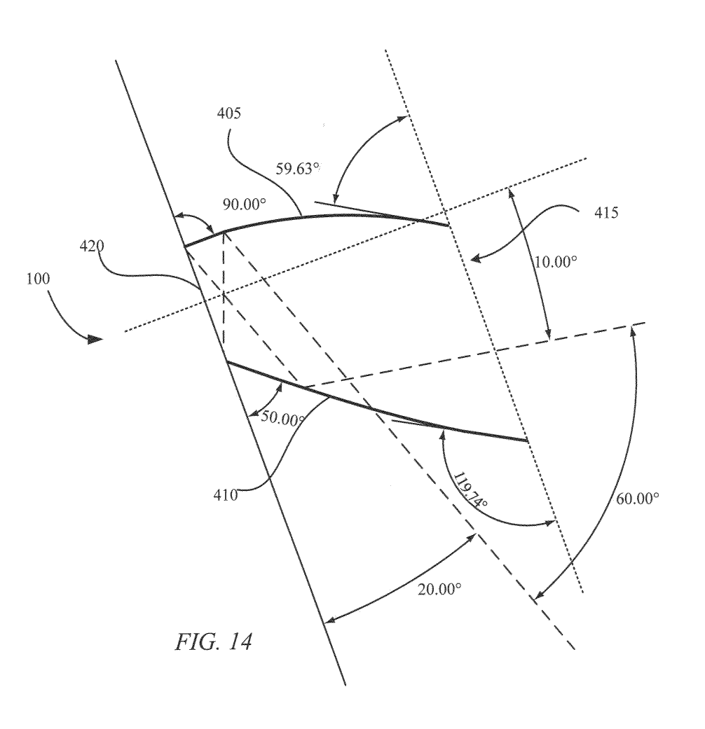

ACPC 100 shown in FIG. 4 is horizontally mounted. ACPC 100 shown in FIG. 14, on the other hand, is almost vertically mounted. That is, exit aperture 420 may be nearly vertically aligned. In other embodiments, an ACPC may be aligned 5°, 10°, 15°, 20°, 25°, 30°, 35°, 45°, 50°, 55°, 60°, 65°, 70°, 80°, 85°, or 90° from vertical. As such, an ACPC may be aligned, for example, on flat surfaces, on the side of a building, or on a pitched roof. The parabolic surfaces 405 and 410 may have any parabolic and/or conic shape. For example, as the angle of the ACPC changes from horizontal as shown in FIG. 4 to nearly vertical as shown in FIG. 14, the parabolic (conic) sections may change depending on the angle of the ACPC. In some embodiments, changes in the parabolic surfaces may be required in order to maintain high concentrator efficiencies.

An ACPC may collect light from the sun without using mechanical and/or actuating tracking devices and still capture a high percentage of sun light as the sun moves through its celestial path with the sun's daily and seasonal variations. In some embodiments, an ACPC may be coupled directly with a light-receiving device. For example, an ACPC may be immovably coupled directly with a solar cell array without requiring the ACPC to rotate or twist to capture more sun light as the sun's path changes. Instead, an ACPC may deliver solar light with at least about 80%, 85%, 90% or 95% efficiency, while maintaining a fixed position relative to any light-receiving device. In some embodiments, an ACPC may provide light at the exit aperture that is substantially perpendicular to the surface of the aperture. Light incident at the exit aperture at such a steep angle undergoes very little reflection allowing more light to exit the exit aperture.

In another embodiment, an ACPC may be coupled with a bioreactor that is used to collect and illuminate biomass within the bioreactor using solar light. In another embodiment, an ACPC may be coupled with a solar cell to collect and focus solar light on the solar cell without tracking devices. In yet another embodiment, an ACPC may be coupled with a solar oven or solar heater without aid of a tracking device. Various other applications for an ACPC may be devised. In such embodiments, the exit aperture of an ACPC may be coupled with an entrance aperture of another optical device or apparatus in order to transmit light there to.

### Illuminating Light Guide

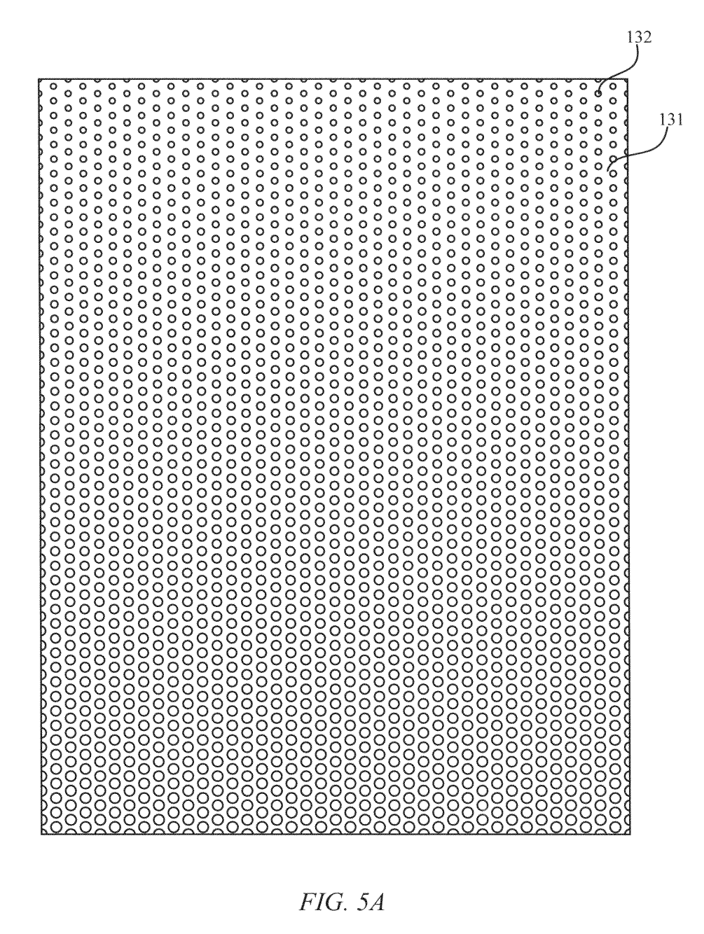

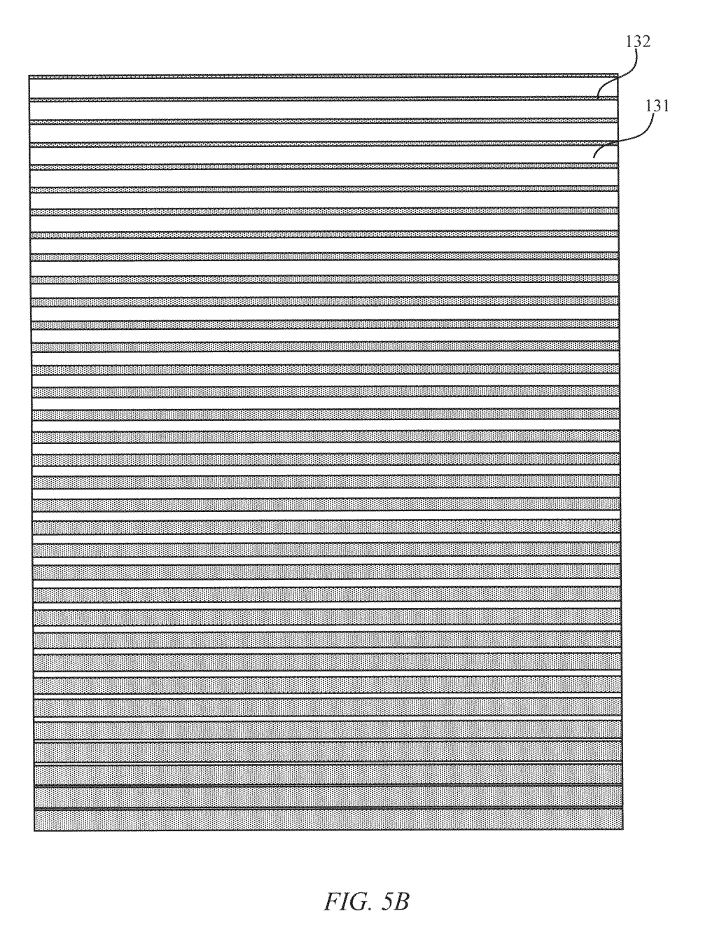

In another embodiment, the present disclosure provides for an illuminating light guide 110 as shown in FIGS. 1, 2, and 3. According to embodiments disclosed herein, illuminating light guide 110 may include two substantially parallel, transmissive, layered sheets 115, 116 that form two surfaces of illuminating light guide 110 and may be coupled with a bottom surface 120 and/or two side surfaces 125. These surfaces may form a trough that may be filled with a clear, transmissive fluid, for example, such as water. Layered sheets 115, 116 may provide uniform transmission of light through the sides of the illuminating light guide. Layered transmissive sheets 115, 116, for example, may include a pattern of air-gaps 131 within a layer in the transmissive sheets 115, 116, as shown in FIGS. 5A and 5B.

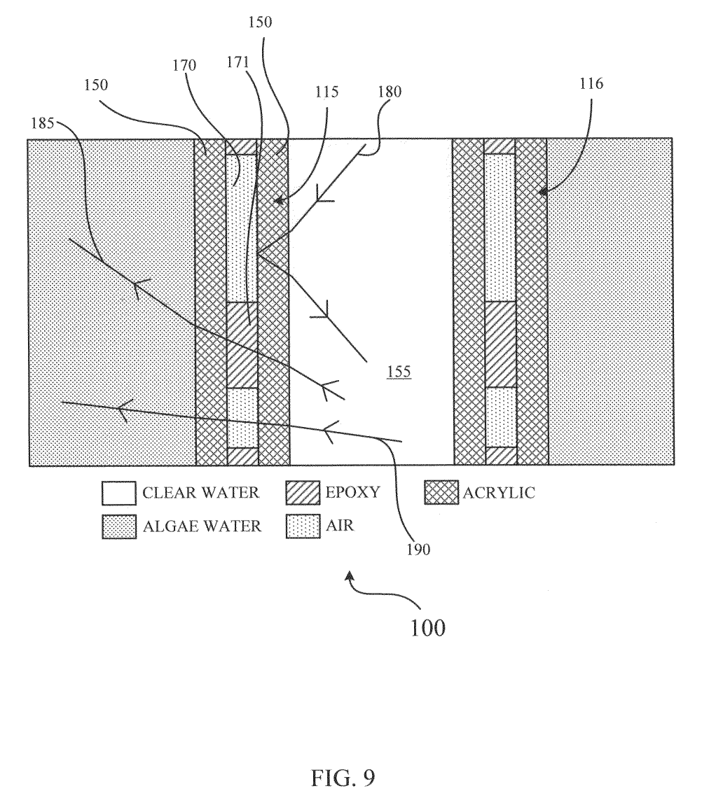

FIG. 9 shows a side view of a trough with transmissive fluid 155 and two layered sheets 115, 116 bounding the trough. Each layered sheet 115, 116 may include two layers of transmissive material 150, such as glass, Plexiglas, acrylic, Lexan, etc. The two sheets may sandwich an air-gap pattern 170 between two outer sheets 150. Following light ray 180, when a layered sheet 115 is submerged in water, each air-gap 170 allows total internal reflection (TIR) at the air-plate interface when light is incident at angles less than the critical angle. Thus, the illumining light guide 110 may receive light through a top surface or an aperture, the light is transmitted through the water, and when the light hits a portion of the of a layered sheet 115 with an air-gap 170 on the other side, TIR occurs when the angle of the incident light is greater than the critical angle as measured from the vertical, and the light is reflected back into the light guide. When light is incident on the layered sheet at an angle less than the critical angle, as measured from the vertical, the light is transmitted through the layered sheet regardless as shown by light ray 190. Now following light ray 185, when the light hits a portion of the layered plate without an air-gap 171, that light is transmitted from the fluid within the light guide to the other side of the layered sheet 115. Accordingly, the higher the percentage of area without an air-gap 171 the higher the percentage of light that will transmit through the layered sheet 115, 116.

The density of the air-gap pattern may vary over the plane of the sheets. For example, a layered sheet may include more air-gaps at the top of the layered sheet 115 and less near the bottom as shown in FIGS. 5A and 5B. As shown in FIG. 5A, the air-gap pattern, for example, may include a pattern of transmissive dots 132 that contact both outer sheets 150 and provide a path for light to transmit from the first outer sheet through to the second outer sheet. Transmissive dots 132 may, for example, comprise epoxy or another adhesive material. Transmissive dots 132 may form a density gradient from the top of the layered sheets 115 to the bottom of the layered sheets 115 with a greater density of transmissive dots at the bottom of the sheet than at the top of the layered sheet permitting greater light transmission near the bottom than near the top. The air-gap pattern, for example, may include a pattern of holes drilled through a third sheet that is in contact with both outer sheets and the second sheet and sandwiched between the first and second sheets. This pattern of holes may form a density gradient from the top of the sheet to the bottom of the sheet with a greater density of holes at the top permitting greater light transmission near the bottom than near the top. Various other air-gap patterns may be used, for example horizontal lines of air-gaps may be used that are thicker at the bottom of the sheet and narrower at the top of the sheet as shown in FIG. 5B. Vertical lines of air-gaps, for example, may also be used that are wider at the top of the sheet and narrower at the bottom of the sheets. In some embodiments, a single layered sheet 115 may be used within an illuminating light guide. In such embodiments, one sheet may be replaced with a reflective material.

Outer sheets may be comprised of thin sheets of material, for example, plexiglass, Lexan or other transmissive materials. For example, outer sheets may be 1/16″, ⅛″, ¼″, ½″, ¾″, 1″, 1-¼″, 1-½″, etc. The side and bottom surfaces may include a reflective material such as mirrors, metal foil, a white surface, etc. that reflect light back into the illuminating light guide. The depth of the trough may be any size or dimension. In one embodiment the depth is about 1.3 meters.

In some embodiments, as shown in FIGS. 1, 2 and 3, an ACPC 100 may be coupled with an illuminating light guide 110. The exit aperture of ACPC 100 may be coupled with the entrance aperture of illuminating light guide 110. Specifically, ACPC 100 may provide light that is relatively perpendicular (i.e., 75°-105°) with the exit aperture of ACPC 100. Light with such a steep angle will undergo totally internal reflection air-gaps within the layered transmissive sheets and will transmit through non-air-gap portions.

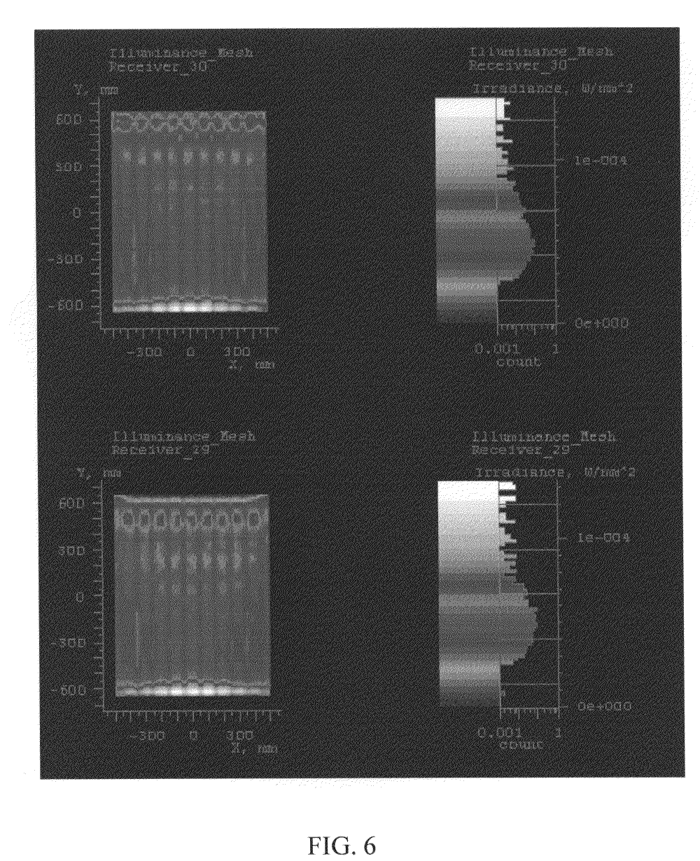

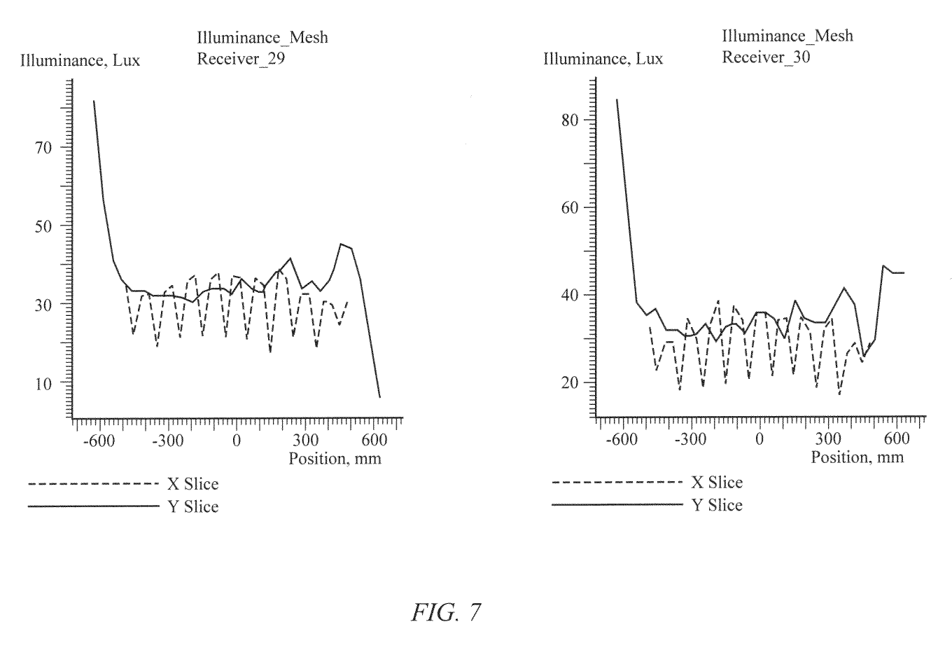

FIGS. 6, 7, 8A and 8B show an example of illumination results for an illuminating light guide and are based on a solar illumination of 1000 watts per square meter of tank surface. This results in the illumination of 15 square meters of area at an average of 30 to 90 Watts per square meter. The light source is a simplified solar model that has a ±30 degree solid angle. The simplified light source used in this design (+/−30 degree cone of light, evenly distributed over the input aperture of the ACPC) was intended to represent solar radiation over both season and time of day, so the uniformity of illumination obtained represents an average over both season and time of day. The layered sheets 115 each send 40% and 42% of the source light into the algae, for a net efficiency of 82%. The efficiency is limited by reflection at the air-water interface, and the finite reflectivity of the CPC reflector and diffuser surfaces. The efficiency of the system (82%) was limited by finite reflectivity of the materials used, and reflection at the air-water interface at the exit aperture of the CPC. The use of an embedded (TIR) air gap (see FIG. 7) as a reflector was a significant factor in obtaining this efficiency.

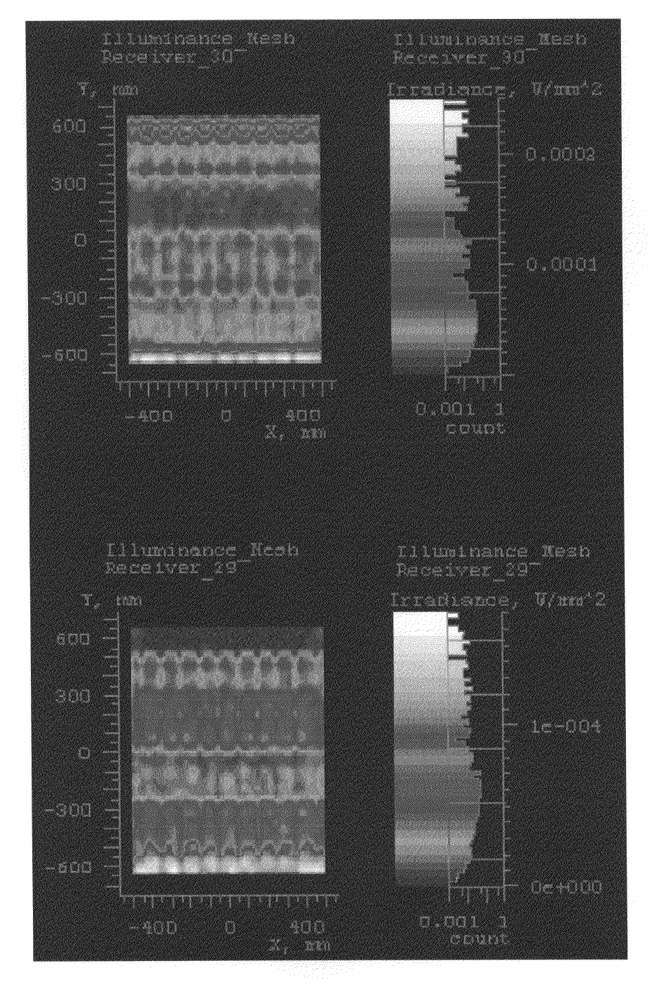

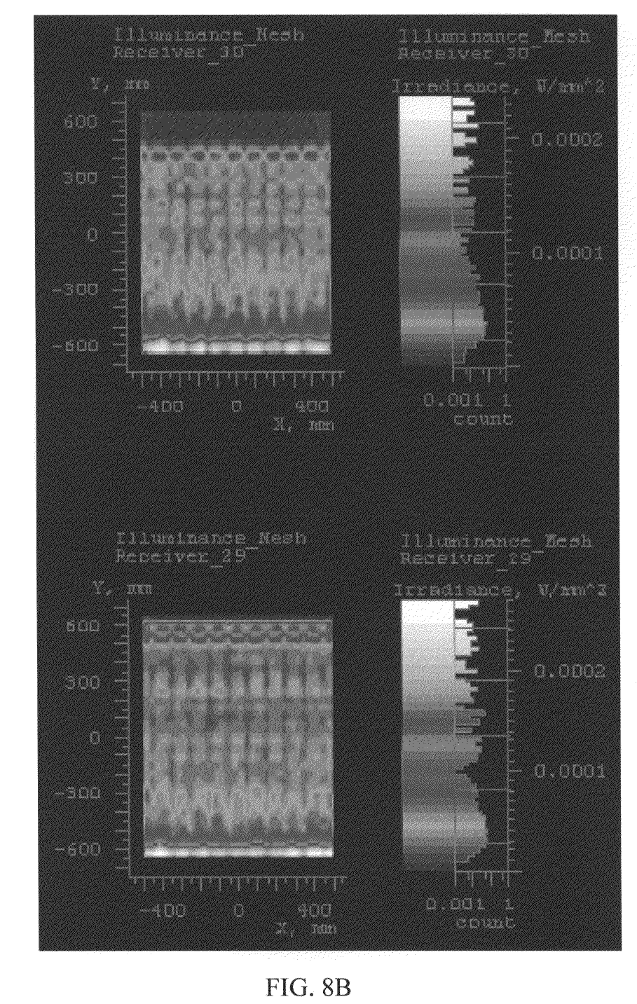

FIGS. 8A and 8B also show the illumination results using parallel light at 20 degrees above and below the CPC axis to illustrate seasonal extremes in the illumination patterns.

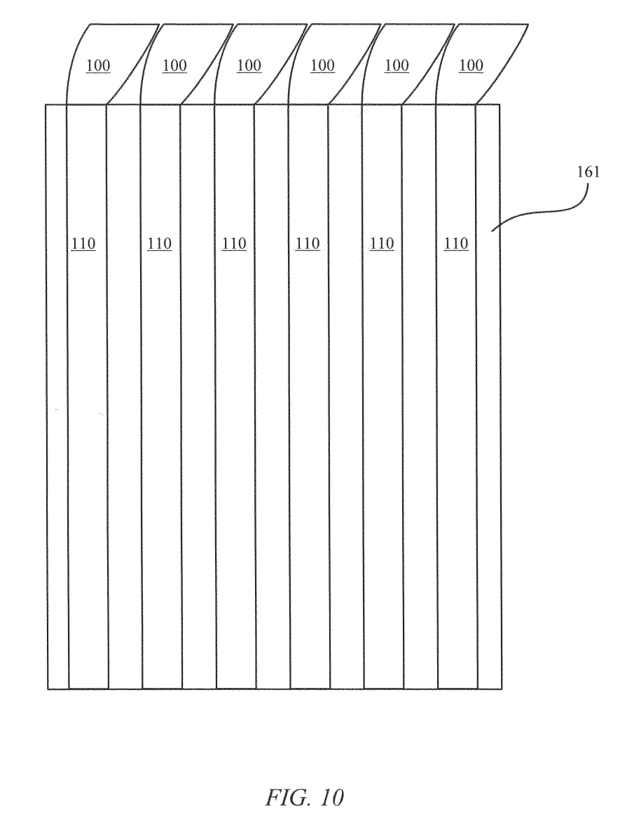

FIG. 10 shows a series of 6 illuminating light guides 110 submerged, for example, in a bioreactor 161. Illuminating light guides 110 are coupled with ACPCs 100.

### Algae Mixer

Algae has a fixed light consumption limit. Algae cannot continuously consume light. For instance, algae may consume a certain amount of light up to a certain limit (4-8 photons); at that point the algae will not consume any more light for a period of time. Accordingly, an algae mixer may be employed to mix algae in a tank (such as a bio reactor) with light sources submerged within, in order to ensure that a high percentage of algae that has not reached its consumption limit is positioned to receive light.

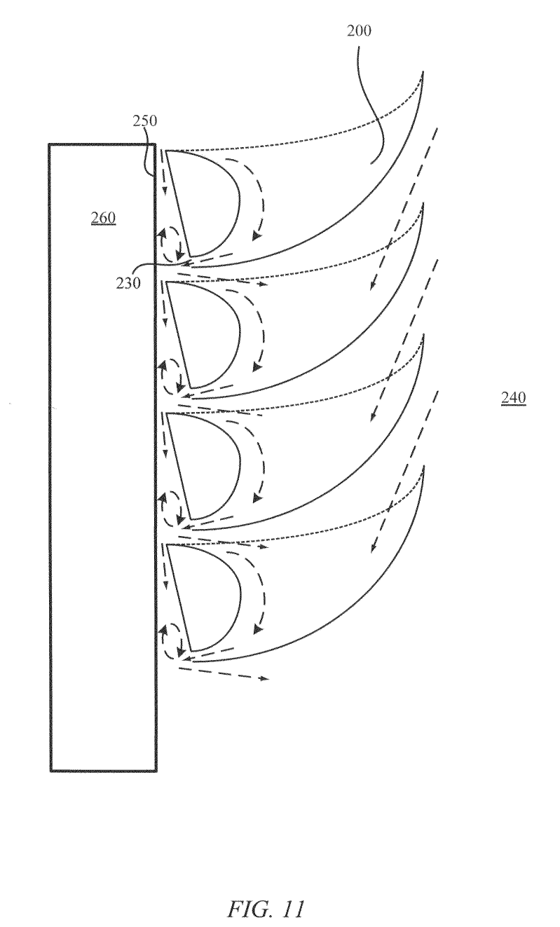

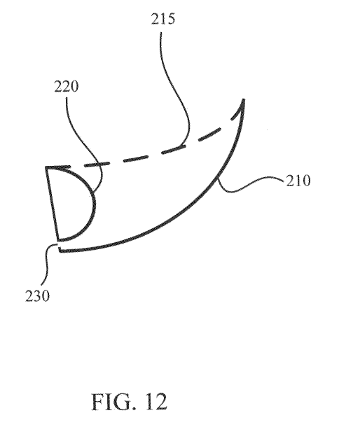

A mixer may include a plurality of scoops 200 as shown in FIG. 11. FIG. 12 shows an individual scoop 200. In some embodiments the mixer may be an algae mixer. The mixer, according some embodiments, may include a series of scoops coupled with an actuating device such as a belt that is configured to move the scoops through a tank of algae. Various other actuating devices may be used, such as cables, wheels, etc. As shown in FIG. 12, each scoop 200 may include at least a concave bottom surface 210, an open top 215, and/or a convex side surface 220. A small opening 230 may be located near the juncture of the convex side surface 220 and the concave bottom surface 210. As a scoop moves through a fluid, the fluid may be forced through small opening 230 as the fluid is compressed by the convex side surface 220 and concave bottom surface 210. The fluid exiting from the scoop through the small opening may exit at speeds greater than the speed of the scoop moving through the fluid. The scoops may be positioned, for example, such that the scoops may move algae in the tank that is originally far from an illuminating surface 240 closer to an illuminating surface 250. Algae mixing may occur by algae being forced through small opening 230 toward an illuminating surface 250.

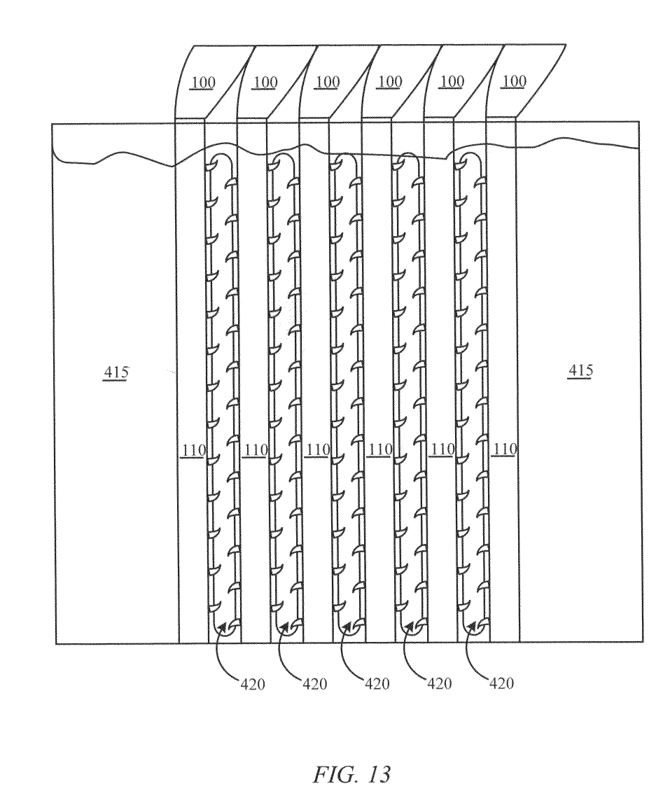

Scoops 200 may be coupled with an actuator that moves scoops 200 through a fluid such as an algae soup. Scoops 200 may be positioned near a light source 260, such as an illuminating light guide. One or more of the scoops may include a squeegee that may be used to clean the surface of the light source. In yet another embodiment, as shown in FIG. 13, a mixer may be placed between two illumination sources, such as illuminating light guides, and the actuator may be configured to move the scoops across a surface of a first illumination source and then across a surface of a second illumination source. For example, the scoops may move up the surface of the first illumination source and down a second illumination source.

FIG. 11 shows the motion of fluid as it interacts with a mixer. Dashed lines show the path of fluid moving as scoops 200 move vertically through the tank. Scoops may be placed close together as shown or further apart.

### Bioreactor

In yet another embodiment, the present disclosure also provides for an algae bioreactor that includes six ACPCs 100, six illuminating light guides 110, an algae tank 415 and five algae mixes 420 as shown in FIG. 13. Any number of ACPCs and illuminating light guides may be used. Illuminating light guides 110 may be immersed within an algae tank 415 that includes algae mixers placed between illuminating light guides 110. An ACPC may be coupled with an illuminating light guide as shown. ACPCs receive light from the sun and direct light into an illuminating light guide. Light from an ACPC enters the illuminating light guide at such an angle that all light rays are greater than the critical angle and thus undergo total internal reflection at the surface of the light guide near an air-gap within the layered sheets. Light may exit the light guide through contacts through the layered sheets 115 and enters algae tank 415. Using layered sheets with a higher percentage of contact near the bottom of the layered sheets, light will transmit through the sheet uniformly into algae tank 415. The algae within algae tank 415 may be mixed using algae mixers described above.

While the principles of the disclosure have been described above in connection with specific apparatuses and methods, it is to be clearly understood that this description is made only by way of example and not as limitation on the scope of the disclosure.

What is claimed is:

1. An asymmetric compound parabolic concentrator comprising:
a first reflective surface having a shape including both a linear portion and a parabolic portion, the first reflective surface having a substantially straight top edge bounding a top portion of the first surface, the first reflective surface having a substantially straight bottom edge bounding a bottom portion of the first surface; and
a second reflective surface having a shape including both a linear portion and a parabolic portion, wherein the parabolic portion of the second reflective portion is different from the parabolic portion of the first reflective surface, the second reflective surface having a substantially straight top edge bounding a top portion of the second surface, and the second reflective surface having a substantially straight bottom edge bounding a bottom portion of the second surface,
wherein the top edge of the first surface and the top edge of the second surface are separated and define an entrance aperture;
wherein the bottom edge of the first surface and the bottom edge of the second surface are separated and define an exit aperture; and
wherein the top edge of the first surface is substantially parallel with the top edge of the second surface, and the bottom edge of the first surface is substantially parallel with the bottom edge of the second surface.

2. The asymmetric compound parabolic concentrator according to claim 1, wherein the concave side of the parabolic portion of the first surface faces the concave side of the parabolic portion of the second surface.

3. The asymmetric compound parabolic concentrator according to claim 1, wherein the asymmetric compound parabolic concentrator is tilted about 40° from vertical.

4. The asymmetric compound parabolic concentrator according to claim 1, wherein one of the first surface and the second surface and the area bounded by the exit aperture are substantially perpendicular.

5. The asymmetric compound parabolic concentrator according to claim 1, wherein the linear portion of the first surface is contiguous with the bottom edge of the first surface.

6. The asymmetric compound parabolic concentrator according to claim 1, wherein the linear portion of the second surface is contiguous with the bottom edge of the second surface.

7. The asymmetric compound parabolic concentrator according to claim 1, wherein the bottom edge of the first surface and the bottom edge of the second surface are coplanar.

8. The asymmetric compound parabolic concentrator according to claim 1, wherein the top edge of the first surface and the top edge of the second surface are coplanar.

9. The asymmetric compound parabolic concentrator according to claim 1, wherein the first reflective surface and the second reflective surface are immovably coupled with a light receiver.

10. The asymmetric compound parabolic concentrator according to claim 1, wherein the entrance aperture is substantially horizontal.

11. A static asymmetric compound parabolic concentrator comprising:
a first reflective surface having a shape including both a linear portion and a parabolic portion; and
a second reflective surface having a shape including both a linear portion and a parabolic portion, wherein the parabolic portion of the second reflective portion is different from the parabolic portion of the first reflective surface, and the parabolic portion of the second reflective portion is concave in the opposite direction as the parabolic portion of the first reflective portion,
wherein the first reflective surface and the second reflective surface are immovably coupled with a light receiver; and
wherein the static asymmetric compound parabolic concentrator concentrates solar radiation regardless of solar variations.

12. The static asymmetric compound concentrator according to claim 11, wherein the top edge of the first surface and the top edge of the second surface are separated defining an entrance aperture.

13. The static asymmetric compound concentrator according to claim 11, wherein the bottom edge of the first surface and the bottom edge of the second surface are separated defining an exit aperture.

14. The static asymmetric compound concentrator according to claim 11, wherein the top edge of the first surface is substantially parallel with the top edge of the second surface, and the bottom edge of the first surface is substantially parallel with the bottom edge of the second surface.

15. An illuminating light guide comprising:
an entrance aperture;
one or more layered transmissive sheets with a portion contiguous with the entrance window, wherein each of the one or more layered transmissive sheets includes a first substantially transparent layer and a second substantially transparent layer sandwiching a patterned air-gap layer; wherein the ratio of the area covered by air-gaps to the area covered by non-air-gaps in the patterned air-gap layer is greater near the entrance window; and
a light conductive material within the illuminating light guide contiguous with a substantial portion of at least one of the one or more layered transmissive sheets.

16. The illuminating light guide according to claim 15, wherein the patterned air-gap layer includes a pattern of substantially transparent dots surrounded by air.

17. The illuminating light guide according to claim 15, wherein the dots near the entrance window have a diameter smaller than dots elsewhere.

18. The illuminating light guide according to claim 15, wherein the patterned air-gap layer includes a pattern of horizontal lines surrounded by air.

19. The illuminating light guide according to claim 19, wherein the thickness of the lines in the pattern of horizontal lines vary according to a vertical gradient.

20. An illuminating light guide comprising:
an entrance window configured to allow light to enter the light guide;
a first transmissive sheet contiguous and substantially perpendicular with the entrance window, wherein the first transmissive sheet includes means for transmitting light from the entrance window through the first transmissive sheet with a substantially uniform vertical intensity gradient; and
a light conductive material within the illuminating light guide contiguous with a substantial portion of the first transmissive sheet.

21. The illumination light guide according to claim 20, further comprising a second transmissive sheet contiguous and substantially perpendicular with the entrance window, and substantially parallel with the first transmissive sheet.

22. The illumination light guide according to claim 20, further comprising a mirror contiguous and substantially perpendicular with the entrance window, and substantially parallel with the first transmissive sheet.

23. An illuminating light guide comprising:
an entrance window configured to allow light to enter the light guide;
one or more layered transmissive sheets with a portion contiguous with the entrance window, wherein each of the one or more layered transmissive sheets includes a first substantially transparent layer and a second substantially transparent layer sandwiching a patterned contact layer; wherein the patterned contact layer is in contact with the first substantially transparent layer and the second substantially transparent layer according to a contact pattern, wherein the contact pattern provides a greater density of contact further from the entrance window and a lesser density of contact near the entrance window; and
a light conductive material within the illuminating light guide contiguous with a substantial portion of at least one layered transmissive sheet.

24. An illuminating light guide comprising:
an entrance window;
a first transmissive sheet contiguous and substantially perpendicular with the entrance window;
a second transmissive sheet contiguous and substantially perpendicular with the entrance window, and substantially parallel with the first transmissive sheet; and
a light conductive liquid secured between the first transmissive sheet and the second transmissive sheet,
wherein the illuminating light guide receives light through the entrance window and transmits the light through the first transmissive sheet and the second transmissive sheet with a substantially uniform vertical intensity gradient through both sheets.

25. An algae mixer comprising:
an actuator; and
a plurality of scoops coupled with the actuator, wherein each scoop includes an opening, wherein when a scoop is moved through a fluid by the actuator a portion of the fluid is captured by the scoop and pressed through the opening at a speed greater than the speed of the scoop moving through the fluid.

26. The algae mixer according to claim 25, wherein each scoop further comprises a concave bottom portion and a convex side portion, wherein the opening is positioned contiguous with a junction of the bottom concave bottom portion and the convex side portion.

27. The algae mixer according to claim 25, wherein the actuator comprises a belt and a motor, and the scoops are coupled with the belt.

28. The algae mixer according to claim 25, wherein the algae mixer is positioned near a light source and at least one scoop includes a squeegee configured to clean a surface of the light source.
